(19) United States
(12) Patent Application Publication
(10) Pub. No.: US 2008/0220492 A1
(43) Pub. Date: Sep. 11, 2008

(54) Stabilization of biological materials through inactivation of metalloenzymes

(76) Inventors: Ernest K. Manders, Pittsburgh, PA (US); Christian D. Manders, Pittsburgh, PA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/900,103

(22) Filed: Sep. 7, 2007

Related U.S. Application Data
(60) Provisional application No. 60/843,436, filed on Sep. 8, 2006.

(51) Int. Cl. C12N 13/00

(52) U.S. Cl. 435/173.2

(57) ABSTRACT

Methods are disclosed for the sterilization of functional biological materials, and for their preservation for shelf storage at uncontrolled temperatures. Biological contaminants are significantly reduced in titer or eliminated while maintaining preservation of functional integrity of sterilized and stabilized products. The sterilized and stabilized functional biological material can be stored at room temperature, thereby making it much more available and easier to use versus, lyophilized, conventional frozen or cold stored biologics. The present invention is further directed to inactivation of metalloenzymes, which are often degradative enzymes in biological systems. Reduction or elimination of the degradative function can be achieved by exposure to ionizing radiation, chemical agents or processes that inactivate the metalloenzymes. Inactivation of metalloenzymes enhances the stability of functional biological materials at ambient temperature.

### CROSS-REFERENCES TO RELATED APPLICATIONS

This application claims the benefit of U.S. Provisional Application Ser. No. 60/843,436, filed Sep. 8, 2006, the content of which is hereby incorporated herein in its entirety by reference.

### BACKGROUND OF THE INVENTION

The present invention relates to the general field of biochemistry and medical sciences, and more particularly to the preservation by irradiation of functioning biological materials, biochemical entities, and biologically active molecules, such as, but not limited to, hemoglobin (within and independent of red cells), antibodies, cytokines, blood components including both formed and non-formed elements, proteins and other cellular components, intact human and animal tissues, and materials acting an antigens for testing for sensitivity and for desensitization, and for vaccines. The present invention is further directed to inactivation of metalloenzymes, which are often degradative enzymes in biological systems, that will lead to stability of otherwise labile functional biological materials, such as antibodies, nucleic acids, proteins, and other molecular entities. Inactivation of metalloenzymes will allow storage of functional biological molecules at room temperature. Inactivation can be achieved by exposure to ionizing radiation or chemical agents or processes that inactivate the metalloenzymes.

Biological materials have been used in a wide range of applications for many years ranging from human and veterinary medical use, diagnostic usage, and in experimental or chemical processes. These materials are often biologically active, meaning that they can perform the same or similar structural, enzymatic, or other molecular functions as they carried out in the organism or plant of origin so that they may be used as diagnostic, preventive, or therapeutic agents. Despite the vast benefits of biological materials, there are risks in using them. These materials can be contaminated with various biological contaminants including viruses, bacteria, yeasts, molds, mycoplasmas and parasites. These contaminants can cause serious health problems if transmitted to a human or animal. They also have the ability to reduce the efficacy or even destroy the materials that they contaminate.

There are many techniques for testing and screening biological materials for biological contaminants or pathogens, but these screening procedures have drawbacks. They are not always reliable, can be costly, and can only test for very specific contaminants. It is much more effective to reduce the risk of wide groups of contaminants. A number of techniques have been utilized to reduce the risk of contamination, including heat treatment, chemical solvents, and radiation. Heat treatment has been shown to often adversely affect the biological activity of the product, reducing the product's efficacy. Chemical solvents, disinfectants, and antibacterial agents have been used to reduce the possibility of contamination, especially the chance of bacterial proliferation after packaging and before use. These solvents and agents can be harmful if they come in contact with humans and often have to be removed from the product prior to use in humans.

Radiation treatment is another way to sterilize a product. Radiation is extremely effective at reducing a wide range of biological contaminants, especially including bacteria and viruses. The published literature in this area teaches that the technique of radiation sterilization is extremely flexible and that one skilled in the art may manipulate certain criteria to include the type of irradiation utilized, oxygen content, pH, rate of irradiation, temperature, solvents, stabilizers, etc. so as to alter the product's reaction to the radiation.

However, most work heretofore in developing radiation treatment protocols has focused on ways to inactivate contaminants, such as bacteria and viruses, generally without regard to persisting structure and biological function. Ideally, such sterilized and stabilized products should be functional, meaning that they can perform the same or similar structural, enzymatic, or other molecular functions as they carried out in the organism or plant of origin so that they may be used as diagnostic, preventive, or therapeutic material. Sufficient ionizing radiation will also inactivate metalloenzymes, which are often the cause of biochemical degradation. By inactivating the metalloenzymes in a functional biological material, the biological function of the irradiated material may be preserved, and that preservation may extend the shelf life of those materials when stored at ambient (room) temperature, for example, from ten degrees Celsius (° C.) to thirty-eight degrees Celsius.

Metalloenzymes form a large class of synthetic and degradative enzymes that are omnipresent in nature. Metalloenzymes are formed from of amino acids joined by peptide bonds and organized into a secondary and tertiary structure with a metal atom or atoms at the active site. In general, metal ions in biological systems function to facilitate electron transport, to bind and activate substrates, to stabilize a protein structure, or to participate in single atom transfer. Metalloenzyme active sites are finely tuned by the surrounding atomic milieu to favor a selected function. Of particular relevance to this application is the observation that a great many degradative enzymes are metalloenzymes. As a class, metalloenzymes are susceptible to ionizing radiation because the metal atoms are relatively large targets for incident radiation and because they are inherently reactive species that may be oxidized and inactivated by the free radicals created by incident radiation in an aqueous environment.

A. Blood and Blood Components

One deficiency in the prior art is in the preparation of blood and blood components for medical use. A wide variety of injuries and medical procedures require the administration of whole blood or a variety of blood components. Every patient does not require whole blood and, in fact, the presence of all of the blood components can cause medical problems. Separate blood fractions can be stored under those special conditions best suited to assure their biological activity at the time of transfusion. For example, when donor blood is received at a processing center, erythrocytes are separated and stored by various methods. Such cells are storable in citrate phosphate dextrose at 4° C. for up to five weeks, generally as a unit of packed erythrocytes having a volume of from 200 to 300 ml and a hematocrit value (expressed as corpuscular volume percent) of about 70 to 90%. Erythrocytes may also be treated with glycerol and then frozen at from 30° to 196° C., and stored for up to seven years in a glycerol solution, but must be kept frozen at low temperatures in order to survive sufficiently for transfusion. Both these methods require careful maintenance of storage temperature to avoid disruption of the desired biological activity of the erythrocytes, and provide a twenty four hour survival time for at least 70% of the transfused cells, which is considered to be an acceptable level for use in transfusion practice in accordance with the American Association of Blood Bank standards.

One known method of storing red blood cells has been the freezing (freeze drying) of red blood cells, since such cells could be stored at room temperature for an extended period of time and easily reconstituted for use in mammals. When RBCs have been lyophilized according to many previous methods, for example, in either an aqueous or phosphate buffered saline (PBS) solution, the reconstituted cells are damaged to the extent that the cells are not capable of metabolizing, and the cell hemoglobin cannot carry oxygen. Glutaraldehyde fixed erythrocytes, which have been lyophilized and reconstituted, have found use primarily in agglutination assays.

It is known that low dose gamma irradiation of blood may be performed to prevent transfusion associated graft versus host disease. Graft versus host disease (GVHD) occurs when donor lymphocytes engraft in a susceptible recipient. These donor lymphocytes proliferate and damage target organs, especially bone marrow, skin, liver, and gastrointestinal tract, which ultimately can be fatal. The disease initially was recognized as a complication of intrauterine transfusion and transfusion to recipients of allogeneic marrow transplant into patients who had received total body irradiation. GVHD also has been seen in other immunologically incompetent patients whose exposure to donor lymphocytes has been from transfusion of cellular blood products or, rarely, a transplanted organ. Finally, the most commonly reported setting for transfusion associated GVHD (TA GVHD) is immunocompetent recipients of blood from biologically related or HLA identical donors. Products implicated in cases of TA GVHD include non irradiated whole blood, packed red blood cells, platelets, granulocytes and fresh non frozen plasma. Frozen deglycerolized red blood cells, fresh frozen plasma and cryoprecipitate have not been implicated.

It is to be understood that the term “blood product” as discussed herein may and usually does include whole blood (and fractions thereof), platelets and/or red cells. The term “white blood cells” as used herein is intended to include the general class of leukocytes, including mononuclear cells and neutrophils, lymphocytes, and any other cells found in the blood, above and beyond red cells and platelets. Also, substantially “cell free” blood products may contain some white cells. At present, gamma irradiation of blood products is the only procedure known to prevent transfusion associated GVHD. The most common irradiation sources are cobalt 60 and cesium 137. Most blood centers rely on a nominal dose of 25 Gy with no less than 15 Gy delivered to any area of the bag for these isotopes to inactivate lymphocytes in cellular products for transfusion.

One of the many problems that plague the use of blood transfusions is the transmission of agents causing infectious disease. Since pathogenic organisms are found in different fractions of whole blood, risks of post transfusion diseases vary depending on the blood product or component used. Several products that are prepared for human, veterinary or experimental use may contain unwanted and potentially dangerous contaminants such as viruses, bacteria, yeasts, molds, mycoplasmas and parasites. A number of these infectious agents are of serious clinical importance in that such agents are not only dangerous to the recipient patients, but can also pose a danger to physicians, and other hospital personnel, handling the blood and blood products. Consequently, it is of utmost importance that such products are determined to be contaminant free before they are used. This is especially critical when the product is to be administered directly to a patient for example in blood transfusions, tissue transplants and other forms of human therapies.

B. Antibodies and Biological Molecules Acting as Antigens

Prior art methods are also deficient regarding preservation of antibodies and biological molecules acting as antigens. Antibodies are now widely used for diagnostic testing. Indeed, many bacteriologic and pathologic diagnoses are rendered by tests depending on the reaction of antibodies with a particular antigen. Such tests include the simple hemagglutination assay used to type blood to the ELISA (enzyme linked immunosorbent assay) test. Storage of antibodies at room temperature would permit their ready use in field hospitals, such as those of military campaigns, so that accurate, rapid diagnosis could be accomplished in an area without established laboratory facilities. Presently, the requirement and need for refrigeration greatly complicates the treatment of patients with antibody preparations. Accordingly, production, transport, and storage of such is unduly expensive and frequently cost-prohibitive.

C. Tissues and Organs for Use in Medical Applications

At present cold storage is the predominant means of preserving allograft tissues and tissue-derived materials for transplantation. Recently there have been a number of infections tracked to banked bone and tendon. Sterilization with irradiation can prevent transmission of both bacterial and viral pathogens. Furthermore, storage at room temperature after irradiation will greatly simplify the preparation, storage, and use of allograft tissues and their derivatives, including bone, collagen and acellular dermis.

Previously, most procedures have involved methods that screen or test products formed from biological materials, biochemical entities and biologically active molecules for a particular contaminant rather than removal of the contaminant from the product. Products that test positive for a contaminant are merely not used. Examples of screening procedures include the testing for a particular virus in human blood from blood donors. However, such procedures are not always reliable. This reduces the value or certainty of the test in view of the consequences associated with a false negative result. False negative results can be life threatening in certain cases, for example in the case of Acquired Immune Deficiency Syndrome (AIDS), for example, when testing for the Human Immunodeficiency Virus (HIV). Furthermore, in some instances, it can take weeks, if not months, to determine whether or not the product is contaminated.

More recent efforts have focused on methods to remove or inactivate contaminants in biological materials, biochemical entities and biologically active molecules before their use. Such methods include heat-treatment, filtration, addition of chemical inactivants and treatment with gamma or other radiation. It is well documented that gamma irradiation is effective in destroying viruses and bacteria. In fact, one author concludes that gamma irradiation is the most effective method for reducing or eliminating levels of viruses. Though biological materials have been sterilized by irradiation, the materials have traditionally been stored refrigerated or frozen following processing. Viral inactivation by stringent heat based sterilization is not acceptable since this could also destroy the functional components of the blood, particularly the erythrocytes (red blood cells) and thrombocytes (platelets) and the labile plasma proteins.

In view of the above, there is a need to provide a method of sterilizing products containing functional biological materials, biochemical entities and biologically active molecules that is effective in removing biological contaminants while at the same time having no adverse effect on the product. Examples of unwanted biological contaminants include viruses, bacteria, yeasts, molds, mycoplasmas and parasites. It is therefore highly desirable to have a safe and economical method and apparatus that will eradicate pathogenic viruses, microorganisms, or parasites present in human whole blood or blood products before such products are infused into a recipient, hence, infecting the recipient with such disease producing agents. At the same time, properly decontaminated blood will also spare the daily threat of infection to hospital personnel who must handle these body fluids. This need is even more acute in a blood bank where donor blood and blood products are stored and processed. It is equally desirable to have a safe and economical method and apparatus that will eradicate pathogenic viruses, microorganisms or parasites present in biological material human or animal tissues (for example, skin, bone, fascia, and tendon) before such tissues are introduced or transplanted into a recipient, thereby preventing infection of the recipient with such diseases. Further, it is desirable to provide a mechanism for the inactivation of metalloenzymes, which are often degradative enzymes in biological systems, particularly functional biological materials. Accordingly, the present invention solves these and other needs.

### SUMMARY OF THE INVENTION

Briefly and in general terms, the present invention is directed to methods for stabilization and preservation of functional biological materials, including the inactivation of metalloenzymes contained in a sample of the material, so as to increase the storage shelf life of the functional biological material at ambient conditions. Such processes of the present invention include exposure to ionizing radiation (such as, but not limited to, gamma radiation), exposure to inactivating agents and/or fluids (such as, but not limited to, binding, oxidation or reduction compounds and supercritical fluids) and exposure to low and/or high temperatures. The present invention further includes methods for preparing products derived from functional material materials (biochemical entities, biologically active molecules) for storage at ambient or room temperature, specifically without the need for pre-irradiation lyophilization (freeze-drying) or post-irradiation freezing or refrigeration.

More particularly, the invention relates to inactivation of potential biological contaminants (for example, viruses, bacteria, yeasts, molds, mycoplasmas and parasites) of compositions including antibodies, peripheral blood cells (for example, red blood cells and platelets), plasma protein fractions (for example, albumin and clotting factors) collected from whole blood (for example, the blood of virally infected persons), body fluids (including but not limited to, urine, spinal fluids, amniotic fluids, and synovial fluids), ex vivo media used in the preparation of anti viral vaccines, and cell culture media (for example, fetal bovine serum and bovine serum) or products derived from such compositions, and solutions of sugars, amino acids, peptides, and lipids for intravenous nutrition. The present invention is further directed to blood based proteins and biologically derived proteins, including, but not limited to monoclonal antibodies, botulinum toxin and plant derived proteins, hemoglobin (within and independent of red cells), antibodies and vaccines. Prior or potential contamination is not a prerequisite for the value of this invention.

Most work in radiation treatment protocols heretofore has focused on ways to inactivate contaminants while rendering the biological material mostly unchanged, in both structure and biological function. The present invention builds on known radiation treatment protocols and teaches that radiation treatments can be effective at not only sterilizing, but also at preserving the function of the biological materials, biochemical entities and biologically active molecules without the need for overtly controlling the temperature of the sterilized product following irradiation. The unique preservation and treatment methods of the present invention render the sterilized product storable at ambient temperature and inactivates the metalloenzymes in the biological materials so as to preserve their functionality, while maintaining the efficacy of the product's biological activity and mechanism of action.

This is a significant advance for a number of reasons. Most importantly, being able to store the processed material at ambient temperature reduces the risk of damaging the material's efficacy if handling procedures are not strictly followed. In many parts of the world, even refrigeration remains a luxury. This has a dramatic impact on the public health of these regions as medicines and vaccines are routinely inactivated due to the inability to keep them refrigerated. The ability to supply a vaccine or biological material that is stable at ambient temperatures in the developing world would have a major impact on human health in those regions. Storage at ambient temperatures also would have a major impact in the developed world by saving money on storage and transportation costs, providing a more convenient and easier to use product in many cases, and making production a less expensive process. Inactivation of the metalloenzymes also allows the biological material to remain functional with a decreased risk of enzymatic breakdown of the material.

One opportunity for use of the present invention is in the preparation of blood and blood components for medical use. The present invention includes methods of sterilizing and storing a whole blood sample or a fraction of biologically derived proteins or structures including formed elements such as cells and tissues, of either plant or animal origin, so that the risk of transmission of infectious diseases, particularly viral diseases, is substantially reduced or eliminated. The present invention further includes methods of preparing biological materials that are inexpensive and easily available to a large percentage of the medical community. Such methods allow for the preservation of a biological material without the need for refrigeration or other treatment that would result in significant additional expense. In addition, the present invention teaches the sterilization and stabilization of biological proteins, such as sterilization of antibodies and other chemical components of the blood, so that the biological proteins may be stored safely at room temperature with a reduced risk of significant loss of biological activity due to degradation from present metalloenzymes and subsequently used with greatly reduced risk of bacterial or specific viral contamination.

Many functional biological materials may be made in accordance with the method of the present invention. Such sterilized products with neutralized degradative metalloenzymes may be used in a method for prophylaxis or treatment of a condition or disease, such that the biological material may be stored at ambient temperature prior to administering an effective amount of the biological material to a patient. Similarly, sterilized and stabilized products formed from irradiated biological materials, biochemical entities and biologically active molecules may be incorporated into diagnostic test methods and kits and for use as elements in industrial and chemical processes. In fact the stabilization of the biological entity through the inactivation of the metalloenzymes will greatly increase the effectiveness for many industrial and chemical processes by reducing unwanted reactions that can reduce the material's effectiveness. Likewise, nutritional solutions containing sugars, amino acids, peptides and lipids may be sterilized and prepared for storage at room (ambient) temperature by this method.

The method of the present invention includes a method of irradiating one or more biological materials, biochemical entities and biologically active molecules so as to preserve its function, inactivate metalloenzymes, and permit storing the resulting sterilized and stabilized product at or about ambient temperature. Functional biological materials suitable for use with the present invention include, but are not limited to, blood or a blood component, such as red blood cells, white blood cells, including monocytes, platelets, clotting factors, immunoglobulins, including mono and polyimmunoglobulins. Likewise suitable functional biological materials include, but are not limited to, animal tissue (including those of mammals and other animal phyla), such as cartilage, bone marrow (including bone marrow cell suspensions), whole or processed ligaments, tendons, nerves, bone (including demineralized bone matrix), grafts, joints, femurs, femoral heads, teeth, skin grafts, heart valves, corneas, arteries, veins, lipids, carbohydrates, collagen (including native, afibrillar, atelomeric, soluble, and insoluble, recombinant and transgenic, both native sequence and modified).

Similarly, the present invention may be applied to non-cellular material, such as proteins (including recombinant and transgenic proteins), proteinaceous materials, amino acids, peptides, sugars, lipids, enzymes (including digestive enzymes such as trypsin, chymotrypsin, alpha-glucosidase and iduronodate-2-sulfatase), antigens, marrow, chitin and its derivatives (including NO-carboxy chitosan—NOCC). The sterilization and stabilization aspects of the methods and resulting products of the present invention include irradiating biological materials, biochemical entities and biologically active molecules by subjecting a measured quantity to a calculated amount of radiation so as to be effective to reduce the risks of viral and bacterial contamination and to reduce the activity of metalloenzymes in the samples thereby sterilizing and stabilizing the biological material.

In the present invention the process of irradiation may be conducted at a variety of temperatures, including but not limited to a common range from room temperature down to extremely cold temperatures including those at the temperature of liquid nitrogen. On rare occasions irradiation with simultaneous heating of the target specimen may be advantageous.

The present invention further provides for the stabilization and preservation of functional and active biological materials through the inactivation of metalloenzymes possessing degradative activity by exposure of the metalloenzymes to ionizing radiation, interacting agents, chemical additives, binding agents, oxidizing or reducing agents, and high or low temperatures. Inactivation of the metalloenzymes, which are enzymes with a metal, usually an ion, linked to its protein component, will reduce the degenerative effects of the biological material. Agents capable of inactivating metalloenzymes include 2-benzyl-3-iodopropanoic acid, phenylethylaminoalanine (PEAA), lysinoalanine, alkali-treated food protein and sulfur amino acids (11), radiation protection thiols and thiol derivatives, ethylenediaminetetraacetic acid (EDTA) (14), N-thiophosphoryl amino acids, dithiothreitol and 2-mercaptoethanol, polycarboxylic acids, and furoic acid and 2-thiophenecarboxylic acid and their derivatives. Supercritical fluid sterilization (for example, ultra-cooled carbon dioxide) may also be used to inactivate metalloenzymes in biological samples. The resulting functioning biologically active material may be stored at room temperature without such conditions as refrigeration, lyophilization, or the addition of stabilizing materials.

Storage of functioning biological materials such as proteins, antibodies, enzymes, hormones, and other entities has heretofore generally required storage at low temperature. The present invention includes inactivation of degradative enzymes, such as metalloenzymes, for example, by ionizing radiation or through exposure to chemical entities, that will inactivate the active site of these degradative proteins, thereby slowing or preventing their destruction of biologically active materials and molecules. The present invention further provides for biologically active and functional materials to be stored at room temperature without significant degradation. This makes useful materials like antibodies, proteins, enzymes, and tissues, etc. available without refrigeration. A room temperature stable biologically active material would have advantages over existing technologies in that it would be less expensive to store, more convenient, and would require fewer preparative steps before usage. In many parts of the world, cold temperature storage of materials is unrealistic and this invention would allow functional biological materials to be used in those regions. The biological material could also be rendered sterile by the incident radiation if desired.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 depicts a table the characteristic of samples tested.

FIG. 2 depicts a chart of Hct vs. Mrads with/without Metheylene Blue.

FIG. 3 depicts a chart of pO₂ VS. Mrads with/without Metheylene Blue.

FIG. 4 depicts a chart of O₂SAT vs. Mrads with/without Metheylene Blue.

FIG. 5 depicts a chart of FO₂Hb vs. Mrads with/without Metheylene Blue.

FIG. 6 depicts a chart of FMetHb vs. Mrads with/without Metheylene Blue.

FIG. 7 depicts a chart of FHHb vs. Mrads with/without Metheylene Blue.

FIG. 8 depicts a chart of Na vs. Mrads with/without Metheylene Blue.

FIG. 9 depicts a chart of WBC vs. Mrads with/without Metheylene Blue.

FIG. 10 depicts a chart of RBC vs. Mrads with/without Metheylene Blue.

FIG. 11 depicts a chart of HGB vs. Mrads with/without Metheylene Blue.

FIG. 12 depicts a chart of HCT vs. Mrads with/without Metheylene Blue.

FIG. 13 depicts a chart of MCV vs. Mrads with/without Metheylene Blue.

FIG. 14 depicts a chart of MCH vs. Mrads with/without Metheylene Blue.

FIG. 15 depicts a chart of PLT vs. Mrads with/without Metheylene Blue.

FIG. 16 depicts a chart of MCHC vs. Mrads with/without Metheylene Blue.

FIG. 17 depicts a chart of RDW vs. Mrads with/without Metheylene Blue.

FIG. 18 depicts a chart of MPV vs. Mrads with/without Metheylene Blue.

FIG. 19 is a photograph of a titer plate depicting an antibody with preservation of biological activity after four weeks from irradiation in accordance with the present invention.

FIG. 20 is a photograph of a titer place depicting a Wright stain of red blood cells after gamma irradiation and storage for four weeks at room temperature.

FIG. 21 is a photograph of a portion of a titer plate depicting an antibody with preservation of biological activity after four weeks from irradiation in accordance with the present invention.

### DETAILED DESCRIPTION OF THE INVENTION

The present invention relates to methods for stabilization and preservation of functional biological materials, including the inactivation of metalloenzymes contained in a sample of the material, so as to increase the storage shelf life of the functional biological material at ambient conditions. Such processes of the present invention include exposure to ionizing radiation (such as, but not limited to, gamma radiation), exposure to inactivating agents and/or fluids (such as, but not limited to, binding, oxidation or reduction compounds and supercritical fluids) and exposure to low and/or high temperatures.

The present invention includes methods for sterilizing and preserving functional biological materials, biochemical entities, and biologically active molecules (biologics) to reduce the level of one or more potential biological contaminants or pathogens therein, such as viruses, bacteria (including intercellular and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for transmissible spongiform encephalo-pathies (TSE). Current storage methods of such biologics typically involve freeze-drying the biologics before sterilization and/or refrigerating the product after sterilization and before transfusion or use in a human body. Certain methods previously disclosed make use of ionizing radiation, but such methods use additional additives and solutions in conjunction with the radiation. For example, some studies have focused on eliminating leukocytes and have generally used low doses of irradiation incapable of effectively inactivating viral burdens in the cells and plasma. There is also no mention in the prior art literature of the ability to store red blood cells at room temperature following irradiation. Our process does not require these additional solutions and reduces the degradation of the biological materials by inactivating the metalloenzymes in the samples. Numerous studies show that red blood cells are not destroyed by the dose needed to inactivate known pathogenic viruses. Therefore, the invention is a simpler way to preserve and sterilize blood before transfusion and a way to make it safer for the recipient and more stable at room temperature.

This invention allows for an easier method of preservation and storage of red blood cells while improving the safety of the material after irradiation. The prior art makes no mention of storage of red blood cells at ambient (for example, room) temperature after irradiation or of the importance of inactivating degradation enzymes that can reduce the effectiveness of biological materials. The prior art also incorporates additional solutions during the irradiation process. It may be beneficial to add a solution that offsets the leakage of potassium from the red blood cells, such as a buffer with or without a potassium-binding agent. The solution may also include additional glucose in the blood bag to supply the red blood cells with additional energy sources. Simply irradiating red blood cells in the proper solution environment within their basic storage bags is suitable for eliminating many known pathogens, inactivating metalloenzymes present in the samples, and allowing the red blood cells to be stored at room temperature.

One aspect of the present invention includes irradiating a sample of a functional biological material (substrate) containing one or more metalloenzymes for a period of time sufficient to provide a sterilizing and/or preserving dose of ionizing radiation, such as gamma radiation from a Cobalt 60 source. Accordingly, such dosage is calculated using ordinary and usual parameters (for example, medium size) known to one having ordinary skill in the art of dosimetry. Irradiation dosages, sufficient to effect sterilization and inactivation of metalloenzymes, are known in the art or may be determined by one having ordinary skill in the art without undue experimentation. Other irradiation variables such as oxygen content, humidity, temperature, time, dose rate, can be altered so as to achieve the optimum dose. One of normal skill in the art will be capable of altering these variables so as to achieve a suitable result. Agents such as those listed here may be added before or after irradiation to further the inactivation of metalloenzymes. The exposure parameters of the inactivation agents are known in the art or may be determined by one having ordinary skill in the art without undue experimentation. After irradiation and/or exposure to an inactivation agent, the finished functional biological material in accordance with the present invention may be stored on a shelf or desk top awaiting its use.

It is contemplated by the present invention that the preserved and stabilized functional biological material made according to teachings of the present invention may be stored at ambient or room temperature for one day, two days, three days, five days, seven days, ten days, twenty days, thirty days, sixty days, one-hundred and eighty days, three-hundred and sixty-five days, two years, and even longer. The storage time at ambient temperature will be dependent on the individual active agents and the type of base material(s) used. The finished functional biological material in accordance with the present invention will be shelf-stable, storable at ambient temperatures, wherein the structural integrity of the base material will be maintained.

### I. CONTAMINANTS

As used herein, the term “biological contaminant or pathogen” is intended to mean a contaminant or pathogen (alone or in combination) that, upon direct or indirect contact with a biological material, may have a deleterious effect on the biological material or upon a recipient thereof. Such biological contaminants or pathogens include various viruses, bacteria (including intercellular and intracellular bacteria, such as mycoplasmas, ureaplasmas, nanobacteria, chlamydia, rickettsias), yeasts, molds, fungi, single or multicellular parasites, prions, agents responsible for TSE, and other agents known to those of skill in the art to be found in or to infect biological materials, biochemical entities and biologically active molecules. Further examples of biological contaminants or pathogens include, but are not limited to, the following: viruses (such as human immunodeficiency viruses and other retroviruses), herpes viruses, filoviruses, circoviruses, paramyxoviruses, cytomegaloviruses, hepatitis viruses (including hepatitis A, B, and C and other variants thereof), pox viruses, toga viruses, Epstein-Barr viruses and parvoviruses; bacteria (including mycoplasmsa, ureaplasmas, nanobacteria, chlamydia, rickettsias), such as Escherichia, Bacillus, Campylobacter, Streptococcus and Staphylococcus; parasites, such as Trypanosoma and malarial parasites, including Plasmodium species; yeasts; molds; and prions, or similar agents, responsible alone or in combination for TSE, such as scrapie, kuru, BSE (bovine spongiform encephalopathy), CJD (Creutzfeldt-Jakob disease), Gerstmann-Straeussler-Scheinkler syndrome, and fatal familial insomnia. As used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in the biological material and/or a recipient thereof.

### II. BIOLOGICS

A. Biological Materials

As used herein, the term “biological material” is intended to mean any substance derived or obtained from a living organism. Illustrative examples of biological materials include, but are not limited to, cells, tissues, blood, blood components, proteins (including recombinant proteins, transgenic proteins and proteinaceous materials), amino acids, peptides (including all natural and synthetic peptides), sugars, lipids, enzymes, including digestive enzymes (such as trypsin, chymotrypsin, alpha-glucosidase and iduronodate-2-sulfatase) immunoglobulins (including monoglobulins and polyimmunoglobulins), botanicals and food. Preferred examples of biological materials include, but are not limited to, ligaments, tendons, nerves, bone (including demineralized bone matrix, grafts, joints, femurs and femoral heads), bone marrow (including bone marrow cell suspensions, whole or processed), teeth, skin grafts, heart valves, cartilage, corneas, arteries, veins, organs (including organs for transplantation, such as hearts, livers, lungs, kidneys, intestines, pancreas), limbs, digits, lipids, carbohydrates, collagen (including native, afibrillar, atelomeric, soluble and insoluble, recombinant and transgenic, both native sequence and modified), enzymes, chitin and its derivatives (including NO-carboxy chitosan “NOCC”), stem cells, islet of Langerhans cells and other cells for transplantation (including genetically altered cells), red blood cells, white blood cells (including monocytes) and platelets.

1. Blood

What has been needed, and heretofore unavailable, is a rapid and safe means of sterilizing blood, blood components and blood products for transfusion while preserving vital function of the formed elements and proteins of the transfusion. Radiation provides not only a means of producing transfusion components of increased safety but it also permits the storage of previously frozen or refrigerated blood components and whole blood at room temperature. Such products and methods of making them will prove to be of great benefit by making the blood supply and the supply of blood components safer and more readily available by virtue of storage at room temperature. It is also anticipated that the shelf life of irradiated blood and blood products will far exceed that of refrigerated blood. Currently about twenty percent of blood transfusion units outdate before transfusion. An increased shelf life will allow use of these outdated units that are wasted at present. This extension of shelf life will therefore have the effect of increasing the blood supply, another salutary effect of the introduction of this new technology.

The present invention is directed to the treatment of tissue to remove biological contaminants, and more particularly to the sterilization and storage of blood and blood components. Moreover, the present invention is directed to the storage of red blood cells at room temperature following irradiation. This invention will permit the transport and storage of banked blood at room temperature.

As used herein, the term “blood components” is intended to mean one or more of the components that may be separated from whole blood and include, but are not limited to, cellular blood components (such as red blood cells, white blood cells, and platelets), blood proteins (such as blood clotting factors, enzymes, albumin, plasminogen, fibrinogen, and immunoglobulins) and liquid blood components (such as plasma, plasma protein fraction “PPF”, cryoprecipitate, plasma fractions, and plasma-containing compositions). As used herein, the term “liquid blood component” is intended to mean one or more of the fluid, non-cellular components of whole blood, such as plasma (the fluid, non-cellular portion of the whole blood of humans or animals as found prior to coagulation) and serum (the fluid, non-cellular portion of the whole blood of humans or animals as found after coagulation).

As used herein, the term “cellular blood component” is intended to mean one or more of the components of whole blood that comprises cells, such as red blood cells, white blood cells, stem cells, and platelets. Viable red blood cells can be characterized by one or more of the following: capability of synthesizing ATP; cell morphology; P50 values; oxyhemoglobin, methemoglobin and hemichrome values; MCV, MCH, and MCHC values; cell enzyme activity; and in vivo survival. Thus, if lyophilized then reconstituted and virally inactivated cells are damaged to the extent that the cells are not capable of metabolizing or synthesizing ATP, or the cell circulation is compromised, then their utility in transfusion medicine is compromised. Conversely, irradiated red cells may still perform biochemical functions without the need for nucleic acid synthetic activity. Unlike most other mammalian cells, red cells are unique in not having a nucleus and therefore they represent more resistant targets in that protein elements may still function after irradiation since they are much smaller that the DNA of the cell and therefore less likely to be inactivated by the incident radiation of a sterilizing beam.

As used herein, the term “blood protein” is intended to mean one or more of the proteins that are normally found in whole blood. Illustrative examples of blood proteins found in animals include, but are not limited to, coagulation proteins both vitamin K-dependent (such as Factor VII and Factor IX) and non-vitamin K-dependent (such as Factor VIII and von Willebrands factor), albumin, lipoproteins (HDL), low density lipoproteins (LDL), very low density lipoproteins (VLDL), complement proteins globulins (such as immunoglobulins IgA, IgM, IgG and IgE). A preferred group of blood proteins includes Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor Ia, Factor IIIa, Factor Va, Factor VIa, Factor VIIa, Factor VIIIa, Factor IXa, Factor Xa, Factor XIa, Factor XIIa, and Factor XIIIa. Another preferred group of blood proteins includes proteins found inside red blood cells (such as hemoglobin), various growth factors, and derivatives of such proteins.

Plasma and serum (the fluid phase after a clot has formed) are components of blood. Numerous proteins and other factors are present and of value in a wide range of medical applications. Antibodies are present in plasma and serum. These may be used in many applications in treating patients (as in the case of gamma globulin, pooled antibodies) and in research. The method of preservation of red blood cells detailed here also applies to the preparation of antibodies and other proteins in that such isolates may be irradiated for the purposes of sterilization and to permit storage at room temperature. This will be of great use in medical settings and in laboratory research. This technology can be applied to antibodies prepared from human blood or from the blood of other animals or from tissue culture fluids from cell cultures producing antibodies. This technology can also be used to prepare sterile viral vaccines for storage at room temperature, thereby greatly simplifying the distribution and administration of vaccines. Additionally red blood cells can be treated and stored at room temperature for use in hemagglutination blood typing tests.

2. Blood Bags

Suitable materials for manufacturing bags and other containers for sterilized and stabilized biological materials, biochemical entities and biologically active molecules include, but are not limited to silicones, plastics, and foils. For example, collapsible, oxygen permeable, silicone bags are suitable for storing irradiated biologics, such as sterilized and stabilized blood products. The use of such bags could be important in converting methemoglobin formed during irradiation to oxyhemoglobin prior to transfusion. An oxygen rich environment could also help make the irradiation more effective at a lower total dose of radiation. Flexible, collapsible bags made of poly(ethylene vinyl acetate) (E.V.A.) plastic are commercially available from Fenwal Division of Baxter Travenol Laboratories, Inc. of Deerfield, Ill.

Red blood cells are often washed to decrease the number of leukocytes in the blood. Leukocytes can elicit an immune response from the recipient of the blood. Due to the risk of bacterial contamination of the blood, these washing processes decrease the shelf life for red blood cells to twenty four hours. Resealing the bag used to store the blood and irradiating it would greatly lengthen the shelf life. Current standards for shelf life following irradiation are twenty eight days from the time of irradiation or the original expiration date on the unit, whichever comes first. This would greatly extend the shelf life of the washed red blood cells.

B. Biochemical Entities

The present invention is directed to preservation by irradiation of functioning biochemical entities and biologically active molecules, such as, but not limited to, hemoglobin (within and independent of red cells), antibodies, peptides (both natural and synthetic), vaccines and other antigens. More particularly, the invention relates to inactivation of potential biological contaminants (e.g., viruses, bacteria, yeast, molds, mycoplasmas and parasites) of compositions comprising antibodies, peripheral blood cells (e.g., red blood cells and platelets), plasma protein fractions (e.g., albumin and clotting factors) collected from whole blood (e.g., the blood of virally infected persons), body fluids (including but not limited to, urine, spinal fluids, amniotic fluids, and synovial fluids), ex vivo media used in the preparation of anti viral vaccines, and cell culture media (e.g., fetal bovine serum and bovine serum) or products derived from such compositions. The present invention further includes methods for preparing whole blood products for storage at room temperature. The present invention is further directed to blood based proteins and biologically derived proteins, including, but not limited to, botulinum toxin and plant derived proteins.

In other embodiments of the present invention, antibodies, clotting factors, growth factors, and other biologically derived proteins, including whole viruses or portions thereof, may be preserved with the irradiation techniques described above for blood and blood components. High dose gamma irradiation will inactivate bacteria and viruses and allow the irradiated materials to be stored at room temperature. This can provide greater availability of biological preparations of vital importance, such as polio vaccine which now must be refrigerated, a barrier to use in such needful areas as Central Africa where there are few facilities with a refrigerator able to store vaccines requiring cold storage.

C. Biologically Active Molecules

As used herein, the term “proteinaceous material” is intended to mean any material derived or obtained from a living organism that comprises at least one protein or peptide. A proteinaceous material may be a naturally occurring material, either in its native state or following processing/purification and/or derivatization, or an artificially produced material, produced by chemical synthesis or recombinant/transgenic technology and, optionally, process/purified and/or derivatized. Illustrative examples of proteinaceous materials include, but are not limited to, proteins and peptides produced from cell culture, milk and other diary products; ascites; hormones; growth factors; materials extracted or isolated from animal tissue or plant matter (including pharmaceuticals such as insulin); plasma and plasma protein fraction (including fresh, frozen and freeze-dried); fibrinogen and derivatives thereof (such as fibrin, fibrin I, fibrin II, soluble fibrin, fibrin monomer and fibrin sealant products); whole blood; protein C; protein S; alpha-1 anti-trypsin (alpha-1 protease inhibitor); butyl-cholinesterase; anticoagulants; streptokinase; tissue plasminogen activator (tPA); erythropoietin (EPO); urokinase; NEUPOGEN (Filgrastim, a granulocyte stimulating factor); anti-thrombin-3; alpha-galactosidase; iduraonate-2-sulfatase; (fetal) bovine serum/horse serum; meat; immunoglobulins (including anti-sera, monoclonal antibodies, polyclonal antibodies and genetically engineered or produced antibodies); albumin; alpha-globulins; beta-globulins; gamma-globulins; coagulation proteins; complement proteins; and interferons.

### III. RADIATION

As used herein, the term “sterilize” is intended to mean a reduction in the level of at least one active or potentially active biological contaminant or pathogen found in the biological material being treated according to the present invention. As used herein, the term “radiation” is intended to mean radiation of sufficient energy to sterilize at least some component of the irradiated biological material. Types of radiation include, but are not limited to, corpuscular (streams of subatomic particles—such as neutrons, electrons and protons); electromagnetic (originating in a varying electromagnetic field—such as radio waves, visible light—both monochromatic and polychromatic, invisible light, infrared, ultraviolet radiation, x-radiation, gamma rays and mixtures thereof); sound waves and pressure waves. Such radiation is often described as either ionizing radiation (capable of producing ions in irradiated materials)—such as gamma rays), and non-ionizing radiation—such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of cobalt or cesium, while UV and X-rays are produced by machines that emit UV and X-radiation, respectively. Electrons are often used to sterilize materials in a method known as “e-beam” irradiation that involves their production via a machine. Visible light, both monochromatic and polychromatic, is produced by machines and may, in practice, be combined with invisible light, such as infrared and UV, that is produced by the same machine or a different machine.

It is known in the art to sterilize a biological material that is sensitive to radiation by irradiating the biological material with radiation for a time effective to sterilize the biological material at a rate effective to sterilize the biological material and to protect the biological material from radiation. See U.S. Publ. No. 2003/0012687 A1 (Macphee et al.), Ser. No. 09/973,958, and U.S. Pat. No. 6,682,695, the contents of which are hereby incorporated herein by reference in their entirety. The present invention, however, builds on such prior art disclosures and describes the first known use of gamma irradiation to sterilize and prepare biologically active molecules, such as a whole blood sample, for use as a biological agent that may be stored at ambient temperature. Because of the risk of the transmission of infectious diseases such as HIV, hepatitis, and other viral diseases, the use of a safe, effective and inexpensive method has become apparent. The only apparent factors limiting the usefulness of this technique are the availability of suitable biologically active molecules (such as blood, blood components, biological proteins, vaccines, viruses and other antigens) and a Cobalt-60 source, or other source of suitable radiation. The low cost of the method of the present invention and the fact that the biologically active molecules are virus-free, and specifically HIV-free, will make this a most attractive means of preparing such biologically active molecules for use in a variety of patients with various needs. Because the process according to the present invention can be often carried out at ambient temperature without requiring the cooling, freezing or chemical treatment of the product containing biologically active molecules before the process is carried out, some of the extra treatment steps that are present in prior art processes are avoided.

By one method of the present invention, gamma radiation is delivered over an extended period of time so as to substantially reduce the damage to the product containing biologically active molecules. Typically, irradiation is carried out for a period of time of not less than ten hours, preferably from about twenty to about forty hours, more preferably from about twenty to about thirty hours. The rate of irradiation is in the range of from about 0.5 kGy/hr to about 3.0 kGy/hr, depending on the product to be sterilized as well as the length of the irradiation time. The total amount of irradiation given is typically in the range of from about twenty to about thirty two kGy, as these levels have been shown to be effective in reducing levels of contaminants such as viruses. Radiation dose delivery as high as 4.0 kGy/min for a time as low as five minutes and higher may be employed for sterilization of biological products with preservation of function and subsequent storage at room temperature.

Preservation by gamma irradiation of biologically active molecules (such as whole blood, blood components, biological proteins and viral entities) has many advantages and makes use of such biologically active molecules a possibility in areas where it is not currently available, such as small hospitals, doctors' offices, and developing countries of the world. The preparation of irradiated whole blood is inexpensive and simple to perform, requiring only basic materials and access to a Cobalt-60 source. Irradiated whole blood can be stored on the shelf at room temperature and does not require liquid nitrogen or low temperature freezer storage. Application of irradiated whole blood requires no thawing, washing or rehydration, as found with other methods of whole blood preservation.

In one method of the present invention, the product containing biologically active molecules may be irradiated in a form containing preferably less than twenty-percent solids. Consequently, certain products may be diluted before irradiation. Treating products in diluted form may also serve to reduce degradation of the product during irradiation. The choice of diluent depends on the nature of the product to be irradiated. For example, when irradiating blood cells one would choose a physiologically acceptable diluent such as citrate phosphate dextrose.

The method of the present invention is useful in treating organic products that are sensitive to irradiation. Such products may be prone to degradation when irradiated by standard methods. However, irradiating sensitive products by the present method would not be expected to be harmful to the products. The method is typically applied to biological products such as blood and blood components, although it is not limited thereto. In cases where living cells (such as blood cells) are to be irradiated, a scavenger may be added to bind free radicals and other materials that are toxic to cells. Suitable scavengers include, but are not limited to, antioxidants, free-radical scavengers, and ligands that stabilize molecules.

Other aspects of the present invention can be practiced by irradiating samples of biologically active molecules for a period of time sufficient to provide a sterilizing dose of radiation. Accordingly, such dosage is calculated using ordinary and usual parameters of dosimetry. Irradiation dosages, sufficient to effect sterilization, are known in the art. Rinsing is not obligatory to practice the invention.

Ionizing radiation may be administered by a source such as a commercial Cobalt 60 or electron beam source. The dose may be selected according to the needs of the material at hand. Bacterial sterilization may be accomplished with reference to tables of radiation sensitivity of bacteria and the need to reduce the bacterial count to less than 10-6 colony forming units. The bioburden present at the start is important for this calculation as is familiar to anyone skilled in the art of radiation sterilization. Biological samples may be sterilized of viruses if an adequate dose of radiation is selected. The common pathogens screened for in donor selection are eliminated by a cumulative dose of 30 kGy or more. Thus, high dose ionizing radiation is capable of sterilizing biological specimens and thereby may eliminate the risk of inadvertent infection by transplantation of allograft and xenograft materials. Appropriate doses may vary according to the needs of a particular situation, varying from 2000 cGy to over 50 kGy, with the most frequent dose being between 3 and 35 kGy.

Radiation may be administered at temperatures from the very cold (liquid nitrogen and dry ice) to room temperature and above. Rates of radiation delivery may vary from about 0.5 kGy/hr to about 4.0 kGy/min for a period of about five minutes to about forty hours. Low temperature renders radiation less effective in inactivating bacteria and viruses. Someone skilled in the art of radiation sterilization knows how to adjust the dose administered to account for the potentially protective effects of low temperature.

Functional biological materials subjected to high dose irradiation may be stored at uncontrolled temperatures, such as room or ambient conditions. Ambient storage conditions in accordance with the present invention include temperatures from about 10° C. to about 38° C. The duration of storage may vary from 5 minutes, to 15 minutes, to 1 hour, to 12 hours, to 1 day, to 7 days, to 30 days, to six months, to 1 year, to 2 years, to 6 years and beyond, and intermediate times in between.

One example of a protocol including the dosage of gamma radiation and time to deliver the dose for irradiation of red blood cells includes:

(i) The dose of irradiation delivered should be 2500 cGy targeted to the central portion of the container and the minimum dose should be 1500 cGy at any other point;

(ii) The time required to deliver the dose should be based on the radiation intensity of the source. The decay of the source should be calculated according to manufacturer's instructions. FDA currently recommends re calibration of the source annually for Cesium 137 and semi annually for Cobalt 60. The procedure for calculating decay, included in the operator's manual for the irradiator, may be referenced in the standard operating procedure (SOP);

(iii) The SOP should indicate the maximum number of units of blood or blood components that can be irradiated at one time. This is a batch and may be dictated by the device manufacturer's procedure and based on the firm's validation data; and

(iv) At no time should the total irradiation dose exceed 5000 cGy to any portion of the container.

As an alternative, blood can be exposed to doses of radiation on the order of 30.0 kGy to sterilize the blood of bacteria, viruses, and other potential pathogens, with subsequent storage at room temperature.

### IV. EXAMPLES

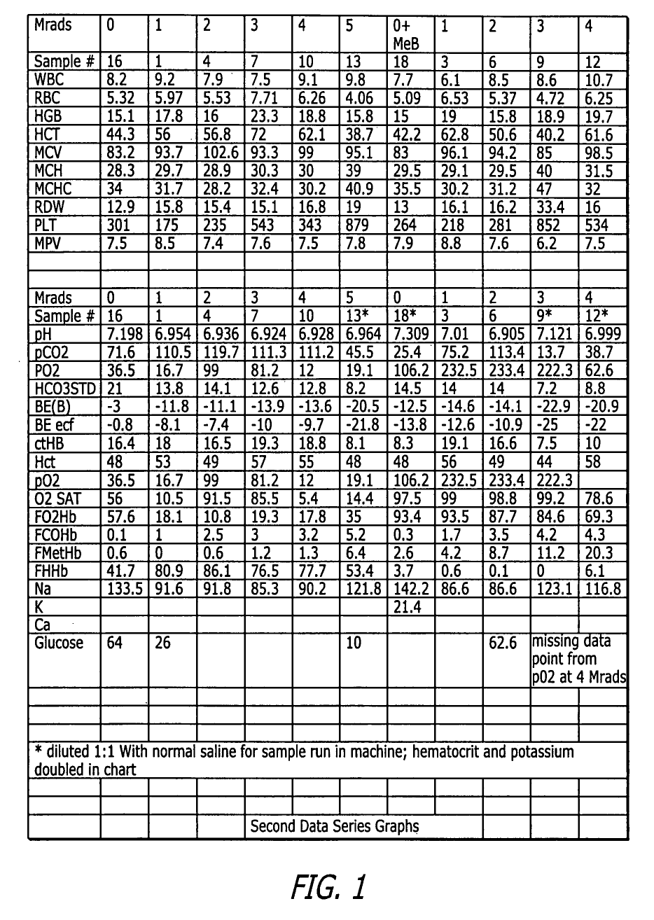

FIGS. 1-21 summarize the experimental data carried out on whole blood using one embodiment of the method of the present invention. Referring now to Table I, blood from a blood bank was irradiated with gamma rays for a total exposure of 30 kGy. The blood was recovered in a USP anticoagulant citrate phosphate dextrose adenine solution (CPDA-1) blood-pack unit (Baxter Healthcare Corporation, Deerfield, Ill.). It was stored at 4° C. for one week after expiration before irradiation. The table lists several characteristics of the blood before and after irradiation. It is noted that the pO₂, HbO₂, and O₂Saturation are significantly reduced after irradiation. The MetHb (methemoglobin) reflecting oxidation of the iron atoms in the hemoglobin is markedly increased. This finding presented a need for finding a way to reduce these changes so that irradiated blood would be more able to readily carry oxygen after transfusion.

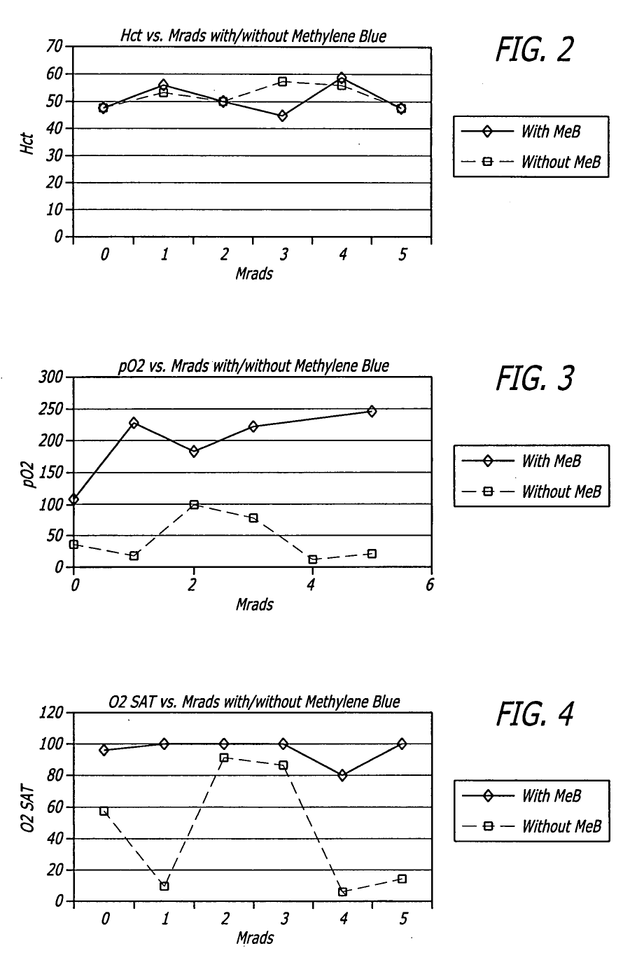

Experimental results demonstrating the effects of irradiation of whole blood in accordance with the present invention can be found in FIG. 1. The protocol for the experimental that generated this data was as follows: Freshly drawn whole blood anticoagulated with EDTA in five milliliter (ml) evacuated tubes was irradiated at room temperature with gamma irradiation in doses varying from zero to fifty kGy in ten kGy steps. One half of the number of tubes served as controls to paired tubes to which 0.01 ml of methylene blue one percent solution (ten mg/ml) was added. Before analysis, oxygen gas was briefly bubbled through the tubes containing the methylene blue to test whether oxyhemoglobin could be formed in high concentration. After irradiation, the blood samples were analyzed in a hospital hematology laboratory using the standard machines of that facility. A number of the results are depicted in FIGS. 2-21.

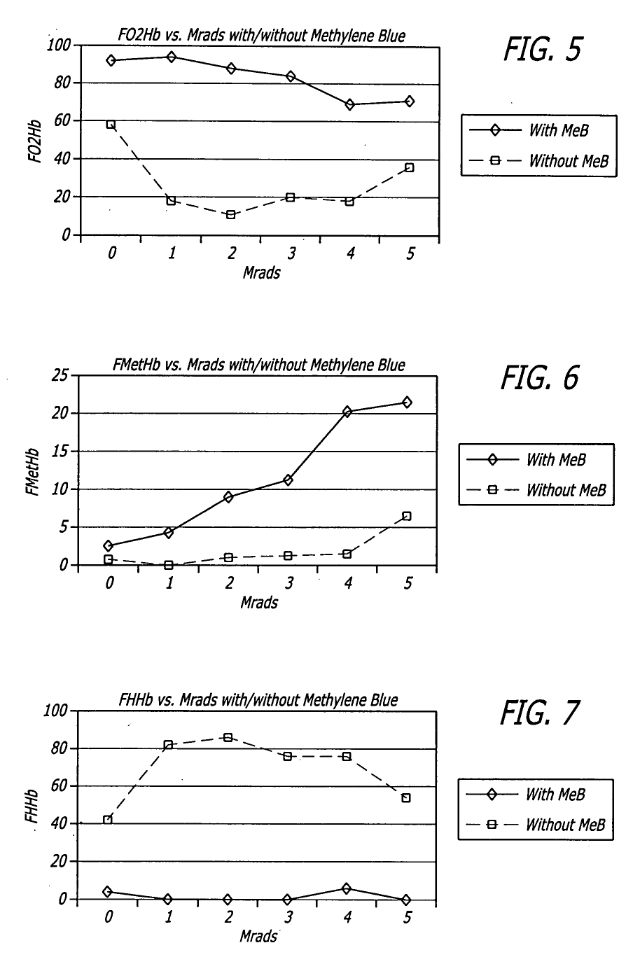

Referring now to FIG. 2, the experiment demonstrates the maintenance of hematocrit after gamma irradiation of whole blood pursuant to the teachings of the present invention. Methylene blue was added as 0.01 ml of a one percent (ten mg/ml) solution per five ml sample tube equivalent to the pharmacologic dose of one to two mg/kg used for treating methemoglobinemia in a patient. As shown in FIG. 3, the partial pressure curve for oxygen showing that after irradiation with methylene blue and exposure to oxygen, there is a higher pO₂ in the sample tubes. Referring now to FIG. 4, the experimental data demonstrates a uniformly high (approaching one hundred percent) saturation of hemoglobin with oxygen in the methylene blue treated irradiated specimens. The saturations at two and three Mrads for the tubes without methylene blue are thought to be due to oxygenation occurring when the blood was agitated in preparation for testing. This demonstrates that methylene blue treated irradiated blood exposed to oxygen can bind the gas. The fraction of hemoglobin bound with oxygen does decrease with increasing radiation dosage, as shown in FIG. 5.

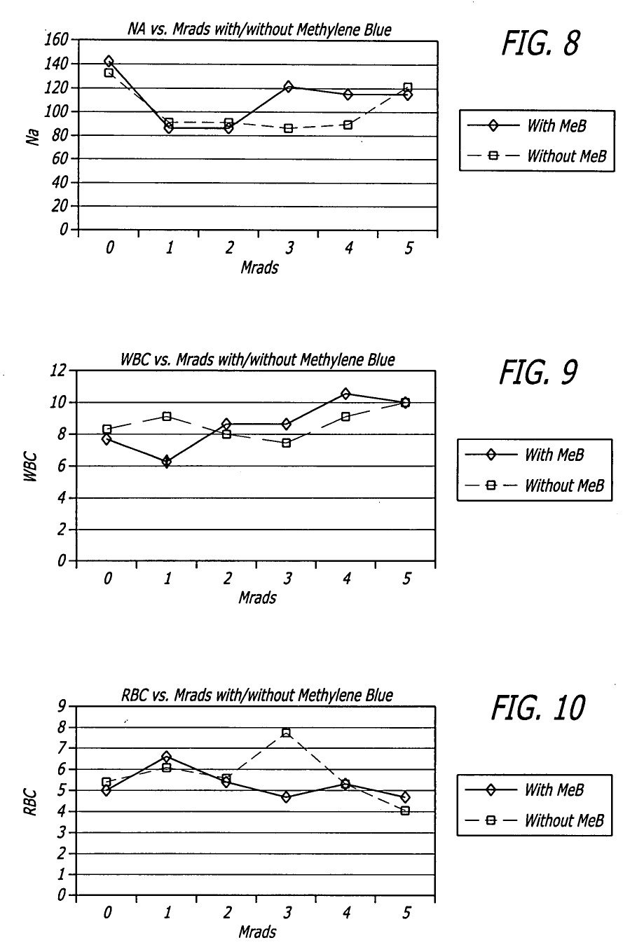

Referring now to FIG. 6, the experiment demonstrates that increasing radiation dose does correlate with increasing methemoglobin content. The MetHb levels for the tubes without methylene blue are unexpectedly low based on other observations, including those of Table I above. Similarly and as shown in FIG. 7, the fraction of hemolysed free hemoglobin is much higher in the tubes without added methylene blue. In addition, sodium concentration in the blood sample tubes demonstrates a relative hyponatremia. Control and methylene blue containing samples show similar values (FIG. 8). Likewise and as shown in FIGS. 9 & 10, white blood cell count and red cell number is not significantly affected by irradiation or presence of methylene blue.

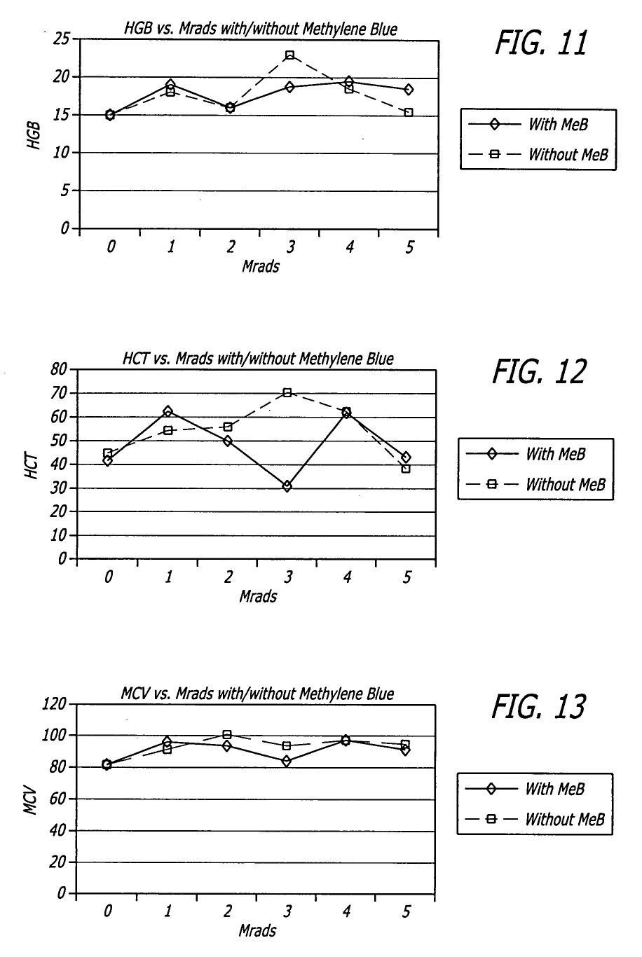

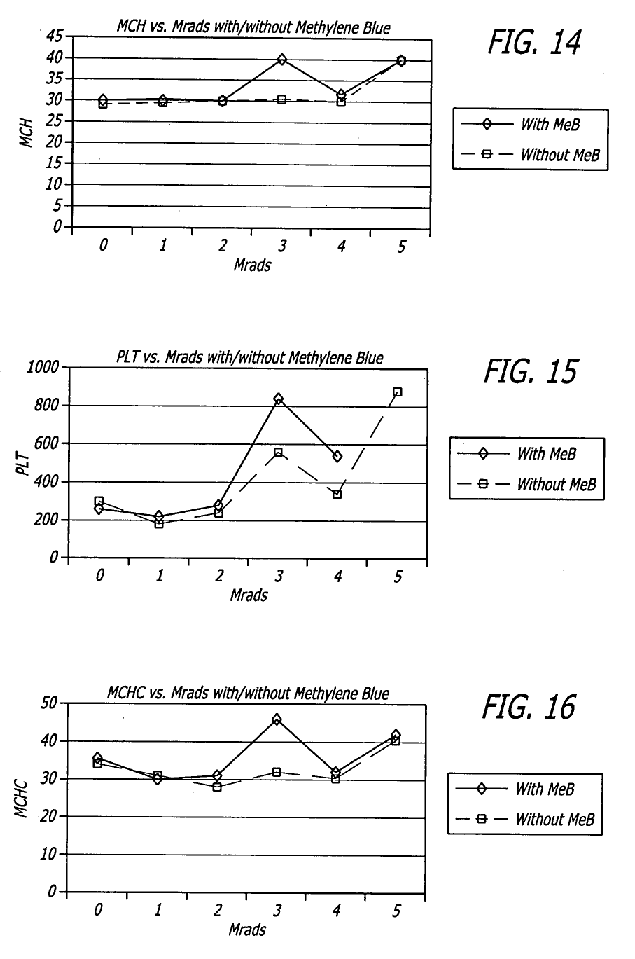

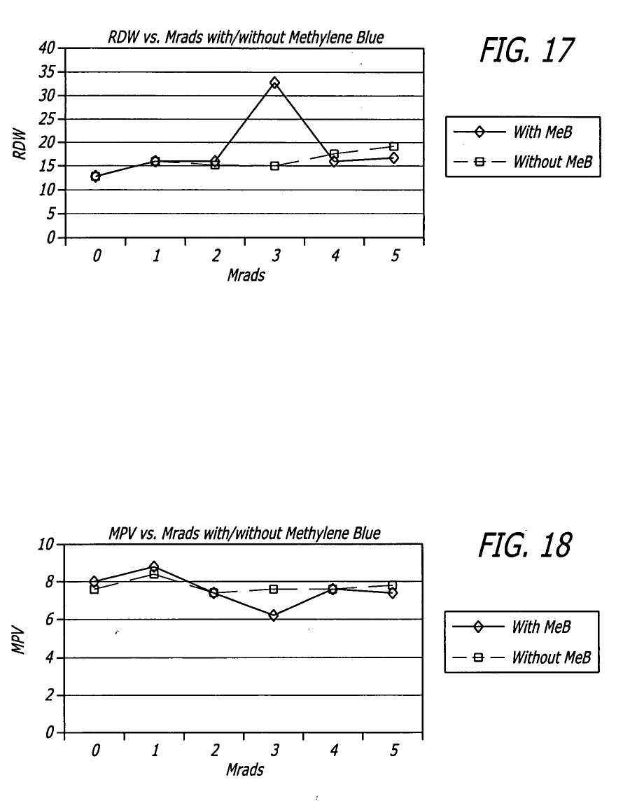

Referring now to FIG. 11, the experiment data shows that hemoglobin content as gm % is affected by irradiation. This is thought to reflect an artifact resulting from radiation-induced change. Others have reported macrocytosis post radiation with increased hematocrit as seen in some samples here (FIG. 12). As shown in FIG. 13, mean corpuscular volume seems to increase with irradiation, as others have noted. FIGS. 14 and 16 show mean corpuscular hemoglobin and mean corpuscular hemoglobin concentration as measured in an experiment conducted in accordance with the present invention. The data also shows that red cell distribution width (FIG. 17) is not dramatically different with the exception of one point that may represent a systematic error of some sort. The data also demonstrates a rise in platelet count (FIG. 15), perhaps as a result of hemoconcentration; whereas, mean platelet volume does not change dramatically under the experimental conditions (FIG. 18).

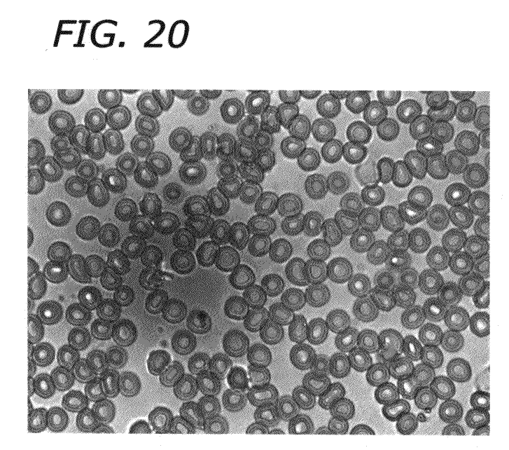

Samples from the irradiated unit of whole blood were examined under the microscope with appropriate hematologic staining and intact red cells were visualized after 30.0 kGys of gamma radiation (FIG. 20). Biochemical analysis revealed methemoglobinemia reversible with standard techniques utilized in medical practice. It appears that the addition of methylene blue and oxygen exposure may be beneficial to convert the iron in the hemoglobin from the oxidized Fe+++ state to the Fe++ state normally present in functioning hemoglobin. Furthermore, a silicone blood bag may help with this conversion by allowing the diffusion of oxygen into the blood and onto the Fe++ atoms before transfusion. As shown in FIG. 20, red blood cells maintained discoid morphology depicted by a Wright stain of red blood cells from a unit of blood collected in a citrate phosphate dextrose adenine (CPDA) blood bag irradiated with 30 kGy and then stored for approximately four weeks at room temperature before study.

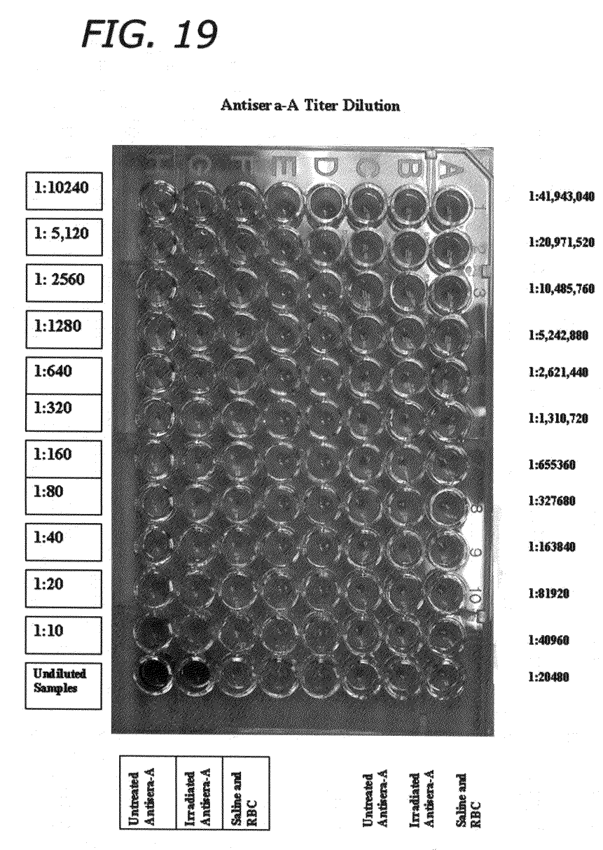

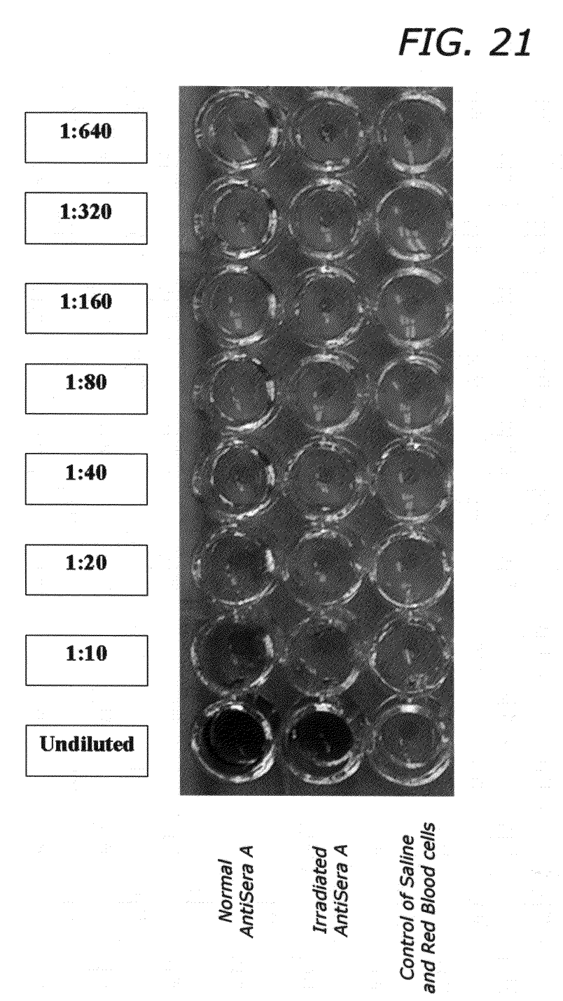

In FIGS. 19 and 21, a demonstration of the ability of anti-A antibody to agglutinate Group A red blood cells is shown. For this experiment, anti-A antiserum was divided into two aliquots. One was stored in the refrigerator; the other was irradiated with 30.0 kGys of gamma radiation and stored at room temperature for one month at room temperature. In this experiment, the ability of the two antisera to agglutinate Group A cells was tested with a serial 1:2 dilution of antibody in physiologic saline in round bottom wells containing Group A cells. The titer of the refrigerated antibody was 1:80 and the titer of the irradiated antibody stored at room temperature was 1:40. The halo of unagglutinated cells is not clearly visible in the black and white reproduction shown in FIGS. 19 and 21.

In one embodiment of the present invention, whole blood from an HIV and hepatitis negative donor may be obtained at the hour of its becoming outdated. Such HIV infected blood may be kept cooled to 40° C. and irradiated with 30 kGy of gamma irradiation. It may then be shipped and stored at room temperature for an extended period of time.

Many useful biologically active molecules and other materials are isolated from biological environments, often cellular environments, such as the cytosol of cells, or suspensions of cells. Such isolations are complicated by the presence of other molecules such as degradative enzymes, of which the metalloenzymes are a broad class. These enzymes are ubiquitous in extractions of biologically active molecules from cells or cell suspensions. They may significantly degrade molecules that are sought after. An example of this problem is well appreciated by those working with the nucleic acid RNA, which is rapidly degraded by the ever present RNAse. This particular enzyme has proven to be a very pesky nemesis of molecular biologists. See Jawad, H. H. and Watt, D. D., “Physical mechanism for inactivation of metalloenzymes by characteristic X-rays,” Int. J. Radiat. Biol., 1986, Vol. 50, No. 4, pp. 665-674; “Letters to the editor—Physical mechanism for inactivation of metallo-enzymes by characteristic X-rays: analysis of the data of Jawad and Watt (Received 12 Jun. 1987),” Int. J. Radiat. Biol., 1987, Vol. 52, No. 4, pp. 651-658; Edited by H. Sigel and A. Sigel, “Metal ions in biological systems,” Volume 30, Metalloenzymes involving amino acid-residue and related radicals, 1994, pp. 2-24; Nisonoff, A.; Hopper, J. E.; and Spring, S. B.; The Antibody Molecule, 1975, pp. 38-43, 264-269, 290-295; each of which are incorporated herein by reference in their entirety.

Metal ions are typically at the active site of these degradative metalloenzymes. The cross sectional area of their electron clouds is very large, making them large targets for incident radiation, such as gamma rays. Exposing metalloenzymes to high dose ionizing radiation (for example, twenty kGy to fifty kGy) may change their valence and the surrounding electronic environment so the enzyme is no longer efficient or even functional in its degradative role. By disabling the degradative function of this class of enzymes, ionizing radiation significantly protects the biological activity of desired entities of use to man. Their expected biological degradation at uncontrolled temperatures (room or ambient) is reduced or eliminated by a loss of activity of the universally distributed metalloenzymes.

Alternatively, metalloenzymes may be inactivated by chemical compounds or interacting agents that bind to key elements of the enzymes as described here and in the references incorporated by reference. The chemical and interacting agents may also inactivate or denature the metalloenzymes, thereby halting the degradative nature of these molecules. Further, chemical additives, such as, but not limited to, binding agents that target the active sites of the metalloenzymes may be used. Similarly, reducing or oxidizing agents of the metals in the enzymes may be added to solutions containing the biologically active materials. When the metalloenzymes are inactivated, the biological materials of interest are not degraded and can maintain form and function for extended shelf life at ambient (room) temperatures (for example, ten degrees Celsius to thirty-eight degrees Celsius) without the need for cold temperature storage, such as refrigeration or freezing, or through some other preservation method, such as, but not limited to, lyophilization.

The present invention further provides for the stabilization and preservation of functional and active biological materials through the inactivation of metalloenzymes possessing degradative activity by exposure of the metalloenzymes to ionizing radiation, interacting agents, chemical additives, binding agents, oxidizing or reducing agents, and high or low temperatures. Inactivation of the metalloenzymes, which are enzymes with a metal, usually an ion, linked to its protein component, will reduce the degenerative effects of the biological material. Suitable high temperatures for exposure to a metalloenzyme so as to inactivate its degradative properties is contemplated by the present invention to be in the range of about forty degrees Celsius to about sixty degrees Celsius, or may be determined by one having ordinary skill in the art without undue experimentation. Similarly, suitable low temperatures for exposure to a metalloenzyme so as to inactivate its degradative properties is contemplated by the present invention to be in the range of about zero degrees Celsius to about ten degrees Celsius, or may be determined by one having ordinary skill in the art without undue experimentation.

Agents capable of inactivating metalloenzymes include 2-benzyl-3-iodopropanoic acid (see Tanaka, Y.; Grapsas, I.; Dakoji, S.; Cho, Y. J.; Mobashery, S., “Conscripting the active-site zinc ion in carboxypeptidase A in inactivation chemistry by a new type of irreversible enzyme inactivator,” Journal of the American Chemical Society, 1994, 116/17, pp. 7475-7480, the content of which is hereby incorporated herein in its entirety by reference), phenylethylaminoalanine (PEAA) (see Friedman, M.; Grosjean, O. K.; Zahnley, J. C., “Inactivation of metalloenzymes by food constituents,” Food and Chemical Toxicology, 1986, 24/9, pp. 897-902, the content of which is hereby incorporated herein in its entirety by reference), lysinoalanine (see Hayashi, R., “Lysinoalanine as a Chelating Agent That Inactivates Metallo-Enzymes,” the content of which is hereby incorporated herein in its entirety by reference), alkalai-treated food protein and sulfur amino acids (see Friedman, M.; Grosjean, O. K.; Zahnley, J. C., “Inactivation of metalloenzymes by lysinoalanine, phenylethylaminoalanine, alkali-treated food proteins, and sulfur amino acids,” Adv. Exp. Med. Biol., Vol. 199, 1986, pp. 531-560, the content of which is hereby incorporated herein in its entirety by reference), radiation protection thiols and thiol derivatives (see Foye, W. O.; Solis, M. C. M., “Inhibition of catalase and lactate dehydrogenase by radiation-protective thiols and thiol derivatives,” Journal of Pharmaceutical Sciences (USA), Vol. 58, 1969, pp. 352-355, the content of which is hereby incorporated herein in its entirety by reference), ethylenediaminetetraacetic acid (EDTA) (see Yang, D.; Wang, J.; Peng, X.; An, L., “Kinetics of inactivation of Ulva pertusa Kjellm alkaline phosphatase by ethylenediaminetetraacetic acid disodium,” J. Enzyme Inhib., Vol. 16, No. 4, 2001, pp. 313-319, the content of which is hereby incorporated herein in its entirety by reference), N-thiophosphoryl amino acids (see Chen, Q.-X.; Lu, H.-Y.; Zhu, C.-M.; Lin, H.-N.,; Zhou, H.-M., “The effects of N-thiophosphoryl amino acids on the activity of green crab (Scylla serrata) alkaline phosphatase,” Biochemistry and Molecular Biology International, Vol. 45, No. 3, 1998, pp. 465-473, the content of which is hereby incorporated herein in its entirety by reference), dithiothreitol and 2-mercaptoethanol (see Zhou, H.-M.; Chen, Q.-X.; Lin, J.-Y.; Zhang, R.-Q.; Zheng, W.-Z.; Zhuang, Z.-L., “Inhibition kinetics of green crab (Scylla serrata) alkaline phosphatase activity by dithiothreitol or 2-mercaptoethanol,” Intl. Journ. of Biochemistry & Cell Biology, Vol. 32, No. 8, 2000, pp. 865-872, the content of which is hereby incorporated herein in its entirety by reference), polycarboxylic acids (see Hegardt, F. G.; Gil., G.; Calvet, V. E., “Inactivation of rat liver HMG-CoA reductase phosphatases by polycarboxylic acids,” J. Lipid. Res., Vol. 24, No. 7, 1983, pp. 821-830, the content of which is hereby incorporated herein in its entirety by reference), and furoic acid and 2-thiophenecarboxylic acid and their derivatives (see U.S. Pat. No. 6,803,379; the content of which is hereby incorporated herein in its entirety by reference). Supercritical fluid sterilization may also be used to inactivate metalloenzymes in biological samples. The resulting functioning biologically active material may be stored at ambient (room) temperature (for example, ten degrees Celsius to thirty-eight degrees Celsius) without further or prior processing, such as refrigeration, lyophilization or the addition of stabilizing materials.

While particular forms of the invention have been illustrated and described, it will also be apparent to those skilled in the art that various modifications can be made without departing from the spirit and scope of the invention. More specifically, it should be clear that the present invention is not limited to the preservation of the specifically recited functional biological materials, biochemical entities and biologically active molecules, but also applies to the preservation of many suitable biologics not specifically named. Likewise, the invention is not limited to any particular metalloenzyme contained in the functional biological material. Further, the present invention is not limited to any particular ionizing radiation or inactivation agent or process. Accordingly, it is not intended that the invention be limited, except as by the appended claims.

We claim:

1. A method of preserving functional biological materials, comprising:
providing a sample containing at least one functional biological material and at least one metalloenzyme;
irradiating the sample so as to preserve the function of the functional biological material and to inactivate a degradative function of the metalloenzyme; and
storing the sample of irradiated functional biological material at ambient temperature.

2. The method of claim 1, wherein the irradiated sample is not refrigerated, frozen or lyophilized prior to use.

3. The method of claim 1, wherein irradiating the sample includes exposing the sample to ionizing radiation so as to stabilize the functional biological material.

4. The method of claim 1, wherein irradiating the functional biological material includes subjecting the functional biological material to radiation in an amount effective to sterilize the functional biological material.

5. The method of claim 1, wherein irradiating the functional biological material includes subjecting the functional biological material to gamma radiation in an amount effective to sterilize the biological material.

6. The method of claim 1, wherein the functional biological material is selected from the group consisting of peptides, blood products, enzymes, toxins, vaccines, growth factors, tissues and antibodies.

7. The method of claim 1, wherein providing a sample containing at least one functional biological material includes providing a blood component selected from the group consisting of red blood cells, white blood cells, monocytes, platelets, clotting factors, immunoglobulins, monoglobulins and polyimmunoglobulins.

8. The method of claim 1, wherein providing a sample containing at least one functional biological material includes providing animal tissue selected from the group consisting of cartilage, bone marrow, bone marrow cell suspensions, ligaments, tendons, nerves, bone, demineralized bone matrix, grafts, joints, femurs, femoral heads, teeth, skin grafts, heart valves, corneas, arteries, veins, organs, carbohydrates and collagen.

9. The method of claim 1, wherein providing a sample containing at least one functional biological material includes providing non-cellular material selected from the group consisting of proteins, proteinaceous materials, enzymes, antigens, amino acids, peptides, sugars, lipids and marrow.

10. A method of preserving functional biological materials, comprising:
providing a sample containing at least one functional biological material and at least one metalloenzyme;
irradiating the sample, wherein the function of the biological material is preserved;
adding an agent to the sample so as to inactivate a degradative function of the metalloenzyme; and
storing the irradiated sample of functional biological material at room temperature.

11. The method of claim 10, wherein the agent is chosen from the group consisting of 2-benzyl-3-iodopropanoic acid, phenylethylaminoalanine (PEAA), lysinoalanine, alkalai-treated food protein, sulfur amino acids, radiation protection thiols and thiol derivatives, ethylenediaminetetraacetic acid (EDTA), N-thiophosphoryl amino acids, dithiothreitol and 2-mercaptoethanol, polycarboxylic acids, furoic acid and 2-thiophenecarboxylic acid.

12. The method of claim 10, wherein irradiating the sample includes exposing the sample to ionizing radiation in the range of twenty kGy to fifty kGy.

13. A method of preserving and sterilizing functional biological materials, comprising:
providing a sample containing a functional biological material, at least one pathogen and at least one degradative enzyme;
irradiating the sample so as to inactivate each pathogen;
processing the sample so as to reduce or eliminate the degradative function of the enzyme; and
storing the sample of irradiated biological material at an uncontrolled temperature, wherein the function of the biological material is maintained.

14. The method of claim 13, wherein processing the sample includes exposing the sample to ionizing radiation.

15. The method of claim 14, wherein processing the sample includes exposing the sample to ionizing radiation in the range of twenty-five kGy to thirty-five kGy.

16. The method of claim 13, wherein processing the sample includes exposing the sample to a supercritical fluid.

17. The method of claim 13, wherein processing the sample includes exposing the sample to a high temperature.

18. The method of claim 17, wherein processing the sample includes exposing the sample to a temperature in the range of forty degrees Celsius to sixty degrees Celsius.

19. The method of claim 13, wherein processing the sample includes exposing the sample to a low temperature.

20. The method of claim 19, wherein processing the sample includes exposing the sample to a temperature in the range of zero degrees Celsius to ten degrees Celsius.
